(19) United States
(12) Patent Application Publication
(10) Pub. No.: US 2007/0121120 A1
(43) Pub. Date: May 31, 2007

(54) Apparatus and method for measuring scleral curvature and velocity of tissues of the eye

(76) Inventor: Ronald A. Schachar, Dallas, TX (US)

Correspondence Address: TIMOTHY F. MILLS, 1177 WEST LOOP SOUTH, SUITE 400, HOUSTON, TX, 77027-9012, US

(21) Appl. No.: 11/601,181

(22) Filed: Nov. 16, 2006

Related U.S. Application Data
(60) Provisional application No. 60/737,180, filed on Nov. 16, 2005.

(51) Int. Cl. G01B 11/02; A61B 3/16; A61B 3/00; A61B 18/18

(52) U.S. Cl. 356/496; 600/398; 606/2; 351/200

(57) ABSTRACT

An apparatus and method for measuring the scieral curvature, velocity and resonant frequency of the tissues of the eye is disclosed. The device preferably comprises a laser with a narrow frequency that is directed at selected tissue of the eye to determine the displacement, phase and frequency shift of the detected light in order to quantify the curvature, and/or velocity and resonant frequency of the selected eye tissue. The curvature of the sclera is used to measure intraocular pressure and the velocity of the surfaces of the eye in response to a vibratory stimulus can also be used to quantify intraocular pressure and stress on different parts of the eye to detect disease such as glaucoma, keratoconus, corneal ectasia, scleral malacia, decline in accommodative amplitude, nuclear sclerosis, macular degeneration and retinopathy.

### CROSS-REFERENCE TO RELATED APPLICATION

Reference is made and priority is claimed from U.S. Provisional Patent Application No. 60/737,180 filed Nov. 16, 2005, entitled “Apparatus and method for measuring velocity of tissue of the eye”, invented by Ronald A. Schacher.

### STATEMENT REGARDING FEDERALLY FUNDED SPONSORED RESEARCH OR DEVELOPMENT

Not applicable.

### BACKGROUND OF THE INVENTION

1. Field of the Invention

The present invention relates generally to a laser device for measuring the scleral curvature and the velocity and resonant frequencies of the tissues of the eye in response to a vibratory stimulus, and more particularly to diagnosis of diseases of the eye.

The measurement of intraocular pressure and the stress on different parts of the eye, are critical physiological variables important in the detection of eye diseases, including ocular hypertension, glaucoma, macular degeneration, retinopathy, the decline in accommodative amplitude, nuclear sclerosis, and presbyopia. Each of these maladies may result in a decline in visual function and in some cases may lead to irreversible blindness.

2. Brief description of the Prior Art

### Measurement of Intraocular Pressure

Methods of determining structural integrity by non-destructive testing have utilized sound waves, electromagnetic radiation and laser beams. The use of lasers generally involves projecting laser emissions onto the surface of a structure, applying a stimulus to the structure to cause it to vibrate, and analyzing the light reflected from the surface. Changes are detected in the reflected light pattern with variations in the frequency and intensity of the vibration stimulus. Based upon the Doppler principle, defects in the structure are determined by detecting a shift in the wavelength of the laser light when it is scattered or reflected from the structure surface. The transmitted light is combined with the scattered light and an interference pattern is produced. The interference pattern is related to the shift in wavelength and therefore to the vibrational velocity of the structure. Thus the use of the laser for non-destructive materials testing utilizes the relationship between resonant frequency and structural integrity. By using laser emissions, defective structures may be determined by comparison of reflected light from a non-defective structure to certain specific changes in reflected light from defective structures.

For example, U.S. Pat. No. 6,915,217, to Springer III et al., discloses a method of remotely inspecting the integrity of a structure, such as an electric light pole, by sonically vibrating the structure and then measuring the response with a remote laser vibrometer or audio detector.

Non-invasive methods to measure the intraocular pressure of the eye have previously been disclosed in the art. Vibration tonometers of the prior art apply variable vibration frequencies to the eye to find the maximum amplitude or resonance point and then interpret the intraocular pressure based on the resonance point. The function of these tonometers is based upon the assumption known as the ‘water-drop’ model, wherein the surface tension of water creates the preferable shape of a sphere, i.e., the human eye, which may be associated with certain resonant frequencies and therefore intraocular pressures.

U.S. Pat. No. 6,673,014, to Badehi et al., departs from the use of the ‘water-drop’ model to determine intraocular pressure. The inventors state that detection of certain resonant frequencies by the ‘water-drop’ model is obscured due to the damping of the surrounding tissue and connective muscles. The undamped natural frequencies of the ‘water-drop’ model converge to a value of zero when the intraocular pressure is zero. However, the inventors determined that the sclera has undamped natural frequencies that are not predicted by the ‘water-drop’ model. In addition, the sclera and/or cornea produce vibratory frequencies with non-zero values for a zero value of intraocular pressure. In one embodiment, their apparatus measures vibratory frequencies of the sclera and/or cornea by exciting the surface with an acoustic stimulus, directing light from an LED toward the excitation point, detecting changes in the angle of the reflected light, and correlating the detected changes with intraocular pressures.

Other types of intraocular pressure measuring devices have involved deforming the front surface of the cornea with a weight, i.e., indentation tonometry (the method of the Schiotz tonometer). The indentation occurs by minimally flattening the cornea with either direct contact, known as applanation tonometry (Goldmann tonometry or piezo-electric pressure transducer), or non-contact via air pressure tonometry (Puff tonometer). These methods have the disadvantage that they depend on mechanical flattening of the surface of the cornea and therefore are subject to error because the measurement depends on corneal thickness, quantification of the corneal flattening, corneal and scleral material properties, and corneal radius of curvature.

A further alternate method for measuring intraocular pressure employs a frequency generator to vibrate the eye between 0 and 4000 hertz and then correlates the peak resonant vibratory frequencies,that must differ by more than 50 Hertz to determine the intraocular pressure of the eye. However, the technique has not been validated, the resolution of the technique is undefined, and the technique involves multiple specialized tonometers to measure the different frequencies.

Therefore, there is a need in the art for a device that can reliably, accurately and objectively measure the velocity of various tissues of the eye that is not limited to invasive techniques, mechanical deformation of the cornea, or the surface methodologies of the prior art to determine the intraocular pressure.

### Measurement of Stress on Different Tissues of the Eye

Accurate measurement of the scleral curvature or stress on the tissues of the eye in the prior art has been problematic. An indirect assessment of stress on the exterior of cornea and sciera is obtained by determining scleral rigidity, which is performed by comparing the difference between intraocular pressure measurements made with an indentation tonometer and an applanation tonometer. However, these measurements are imprecise due to the variables described above and do not accurately quantify the stress on each tissue. Another method for measuring corneal stress involves determining the amount of corneal flattening using air pressure tonometry. This technique is also subject to error because the measurement depends on variables such as corneal and/or scleral thickness, quantification of the corneal or scleral flattening, corneal and scleral material properties, and corneal radius of curvature.

There presently is no non-invasive apparatus or method in the prior art that can accurately measure the scleral curvature or the stress on the interior tissues of the eye, including but not limited to the optic nerve, the crystalline lens, the retina and the retinal blood vessels. Therefore, there is a need for a non-invasive apparatus that can reliably, accurately, precisely and objectively measure the scleral curvature and the stress of the different tissues of the eye.

### SUMMARY OF THE INVENTION

The present invention disclosed herein comprises an apparatus and method to accurately, precisely and objectively measure intraocular pressure and stress on tissues of the eye by utilization and detection of laser emissions.

A preferred embodiment of the present invention comprises utilization of a laser to remotely assess scleral curvature and the microscopic velocity and resonant frequencies of the different parts of the eye in response to a vibratory stimulus.

The laser of the preferred embodiment emits a specific wavelength of light within a narrow frequency to measure the curvature and velocity of each tissue of the eye. To determine the velocity of a tissue of the eye in the preferred embodiment a vibratory stimulus is applied. The precision of the laser detection system in the preferred embodiment is dependent on the selected wavelength of the laser and is most preferably in the 10 nanometer per second range.

The apparatus of the preferred embodiment permits scleral curvature measurement in the nanometer range and the response to vibratory stimuli in the 0 to 2000 hertz range. The surfaces of the tissues of the eye typically move in the micron per second range. Scleral curvature measurement in the preferred embodiment is derived by detecting reflected laser radiation from two to an infinite number of points.

To elicit vibration frequencies in the tissues of the eye for laser detection, stimuli are applied by direct contact, indirect contact, or by remote methods. A direct stimulus to the eye may be provided by contacting a piezoelectric frequency generator to the cornea or sclera. For indirect contact, a piezoelectric frequency generator may be placed on the eyelid or on the skin covering one of the bones of the head. Alternatively, the piezoelectric frequency generator may be held in the hand of the subject or placed on any other part of the body. For remote stimulation the vibratory stimulus may be generated by an audio speaker, piezoelectric frequency generator, or produced by modulating the amplitude and frequency of a stream of air that is, or is not, directed at the eye.

The preferred embodiment of the apparatus of the present invention comprises generally a stimulus generator, a laser emitting a beam of known wavelength, and a detector apparatus. The laser is directed to a beam splitter dividing the emitted beam into a reference beam and a measuring beam. The measuring beam is directed through a biomicroscope, or other suitable device, then focused on the target tissue. The backscattered light reflected from the tissue is collected through the biomicroscope, or other suitable device, and directed to impinge upon a detector. Simultaneously, the reference beam is directed to impinge on a detector. The detector output compares the backscattered light to the reference beam to determine the frequency and phase-shift of the backscattered light, and then calculates the velocity and resonant frequencies of the target tissue. Correlation between the detected velocity and resonant frequencies of the tissues, and known parameters for normal and disease states, aids in diagnosis.

To redress the deficiencies of the mechanical tonometric methods of the prior art, it is a primary object of the present invention to provide a device that is capable of measuring the intraocular pressure and stress on different tissues of the eye without the need for deformation of the tissues.

It is a further object of the present invention to provide a device for measuring the intraocular pressure of the eye by measuring scleral curvature and/or the velocity and resonant frequencies of the surfaces of the eye in response to a vibratory stimulus.

It is a further object of the present invention to measure scleral curvature and/or the velocity and resonant frequencies of the corneal or scleral surfaces, the iris, iris blood vessels, surfaces of the crystalline lens, the walls of the retinal blood vessels, the surface of the retina, Bruch's membrane, lamina cribosa, and other tissues of the eye with a laser.

It is a further object of the present invention to determine the stress on the cornea, the sclera, the iris, the iris blood vessels, the crystalline lens, walls of the retinal blood vessels, the retina, Bruch's membrane, lamina cribosa, and other tissues or interfaces of the eye.

It is a further object of the present invention to measure the velocity of the surface of cornea and/or sclera, the iris, the surfaces of the crystalline lens, the walls-of the retinal blood vessels, the surface of the retina, Bruch's membrane, lamina cribosa, and other tissues of the eye by measuring the phase and/or frequency shift of the laser beam that occurs following reflection of the laser from these tissues of the eye.

It is a further object of the present invention to measure the velocity of the surface of the cornea, the sclera, the iris, the iris blood vessels, the surfaces of the crystalline lens, the walls of the retinal blood vessels, the surface of the retina, Bruch's membrane, lamina cribosa, and other tissues of the eye by measuring the Doppler shift of the laser beam following reflection of the laser from the tissue of the eye being subjected to a vibratory stimulus.

It is a further object of the present invention to measure the difference between the velocity of the corneal and/or scleral surfaces, the iris, the iris blood vessels, the surfaces of the crystalline lens, the walls of the retinal blood vessels, the surface of the retina, Bruch's membrane, lamina cribosa, and other tissues of the eye that are associated with a vibratory stimulus.

It is a further the object of the present invention to scan the surface of the cornea, the sclera, the iris, the iris blood vessels, the surfaces of the crystalline lens, the walls of the retinal blood vessels, the surface of the retina, Bruch's membrane, lamina cribosa, and other tissues of the eye to determine the variation in velocity of different areas of these tissues in response to a vibratory stimulus.

It is a further object of the present invention to measure the velocity of the surface movements of the cornea, the sclera, the iris, the iris blood vessels, the surfaces of the crystalline lens, the walls of the retinal blood vessels, the surface of the retina, Bruch's membrane, lamina cribosa, and other tissues of the eye that are associated with a vibratory stimulus consisting of a single and/or multiple frequencies extending from 0 to 10 Megahertz, preferably from 5 to 10 megahertz.

It is a further object of the present invention to measure the material properties of the cornea, the sclera, the iris, the iris blood vessels, the surfaces of the crystalline lens, the walls of the retinal blood vessels, the surface of the retina, Bruch's membrane, lamina cribosa, and other tissues of the eye that are associated with a vibratory stimulus by measuring the amplitude, frequency and velocity of the response of these tissues to a vibratory stimulus.

It is a further object of the present invention to measure the velocity of the tissues of the eye by transmitting a laser beam and/or beams through a biomicroscope (slit lamp), surgical microscope, or stereo-microscope.

It is a further object of the present invention to measure the velocity of the surface of the eye by transmitting a laser beam and/or beams through fiber optics.

The foregoing has outlined rather broadly the features and technical advantages of the present invention so that those skilled in the art may better understand the detailed description of the invention that follows. Additional features and advantages of the invention will be described hereinafter that form the subject of any claims of the invention. Those skilled in the art should appreciate that they may readily use the conception and specific embodiment disclosed herein as a basis for modifying or designing other structures for carrying out the same purposes of the present invention. Those skilled in the art should also realize that such equivalent constructions do not depart from the spirit and scope of the invention in its broadest form.

Before undertaking the Detailed Description of the Invention below, it may be advantageous to set forth definitions of certain words and phrases used throughout this patent document: the terms “include” and “comprise” as well as derivatives thereof, mean inclusion without limitation; the term “or,” is inclusive, meaning and/or; the phases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, those of ordinary skill in the art should understand that in many, if not most instances, such definitions apply to prior uses, as well as future uses, of such defined words and phrases.

### BRIEF DESCRIPTION OF THE DRAWINGS

For a more complete understanding of the present invention and its advantages, reference is now made to the following description. taken in conjunction with the accompanying drawings, in which like reference numerals represent like parts:

FIG. 1 illustrates a schematic diagram of an embodiment of the laser eye velocitometer in accordance with the present invention.

FIG. 2 is a flow diagram of an embodiment of the laser eye velocitometer in accordance with the present invention.

FIG. 3 shows the experimental results of tests on a mammal eye measuring the velocity of the corneal surface in accord with the present invention and as further described herein.

FIG. 4 shows the experimental results of tests on a mammal eye measuring the curvature of the sclera and its correlation with the intraocular pressures in accord with the present invention and as further described herein.

### DETAILED DESCRIPTION OF THE INVENTION

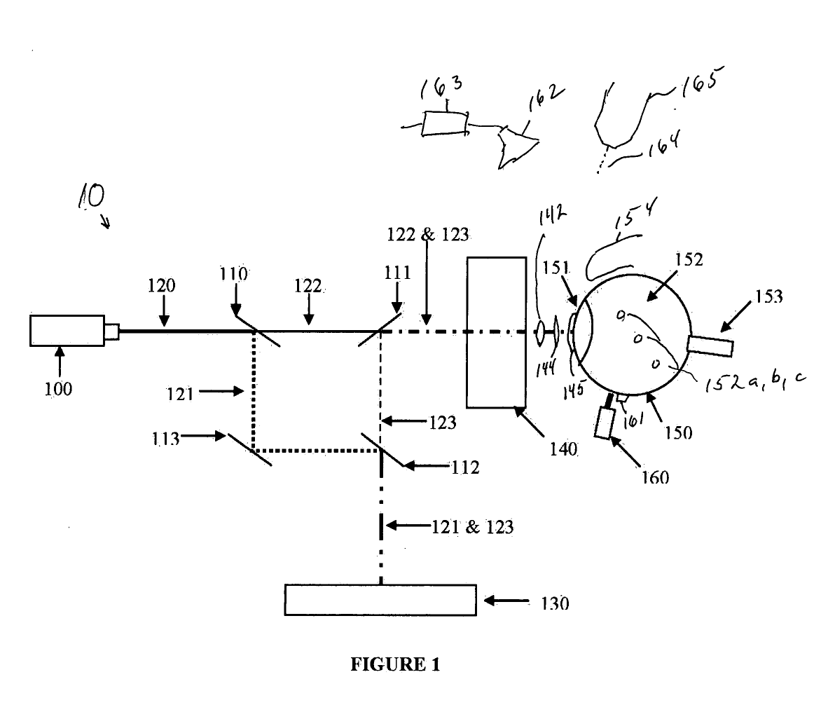

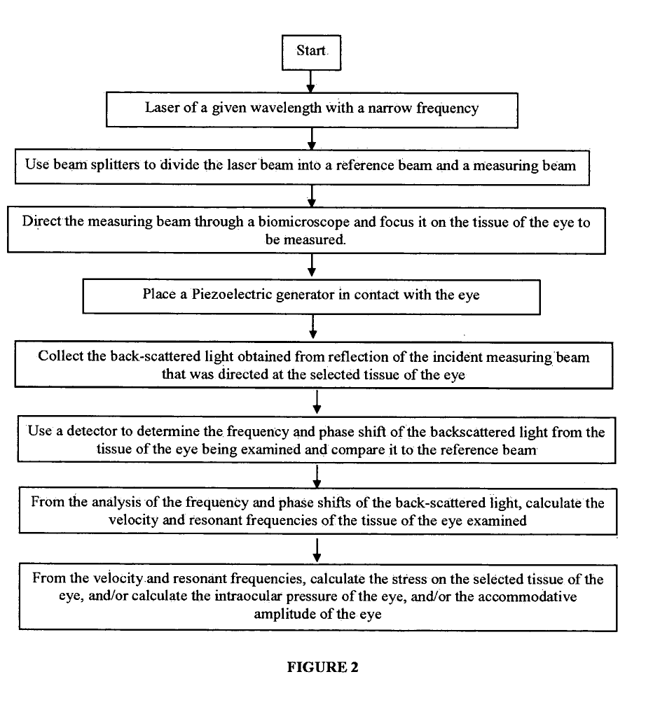

FIGS. 1 and 2 and the various embodiments used to describe the principles of the present invention are by way of illustration and should not be construed in any way to limit the scope of the invention. Those skilled in the art will understand that the principles of the present invention may be implemented in a variety of suitably arranged laser, vibration stimulus, mirror and detector assemblies.

FIGS. 1 and 2 illustrate a schematic diagram of a preferred embodiment of the laser-eye velocitometer 10 of the present invention for measurement of tissue velocity or the curvature of the sclera. The laser, 100, is a laser of a given wavelength, e.g. a Helium Neon laser with a wavelength of 633 nm that has a narrow frequency. The laser beam, 120, emitted from the laser is directed to a beam splitter, 110, to produce a plurality of beams, such that the laser beam, 120 is divided into a reference beam, 121, and a measuring beam 122.

The reference beam 121 is reflected from mirror 113 and then reflected from mirror 112 to join the backscattered light 123 that is reflected from the tissue of the eye selected for measurement, so that both beams, 121 and 123, impinge on the detector, 130.

The measuring beam 122 is directed through a biomicroscope 140 and focused on a tissue of the eye 150, for example the cornea 151, or the sclera 152 or the optic nerve 153. The backscattered light 123 is collected through the biomicroscope 140 and directed by mirror 111 to join the reference beam 121 and impinge on the detector 130.

The detector 130 compares the backscattered light 123 to the reference beam 121 and determines the scleral curvature and/or the frequency and phase shift of the backscattered light 123 and the velocity and resonant frequencies of the tissue of the eye 150. For the frequency and phase shift a vibratory stimulus induced by a piezoelectric frequency generator 160 is applied to the eye.

In another embodiment, laser 110 is a diode laser. In further embodiments the laser 110 may be a tunable laser, or any laser that emits a specific wavelength selected from the spectrum that extends from the ultraviolet to the infrared.

In a further embodiment the measuring beam 122 is directed through a surgical microscope 140.

In a further embodiment the measuring beam 122 is directed through a fiber optic bundle 140.

In a further embodiment the measuring beam 122 is directed through a retinal camera 140.

In a further embodiment, the position of the measuring beam 122 is controlled by steering optics 142 so that location of impingement of the measuring beam 122 on the selected tissue of the eye 150 can be altered without changing the position of the biomicroscope 140.

In a further embodiment, the path of the reference beam 121 is controlled by steering optics 143.

In a further embodiment, the measuring beam 122 is directed at one tissue of the eye and the reference beam 121 is directed at a different tissue of the eye to determine the differential velocity and differential resonant frequencies of the two different tissues of the eye.

In a further embodiment to enable measurement of tissues within the eye, a concave lens or a +90 diopter lens 144 may be placed between the biomicroscope 140 and the eye. Alternatively, a gonio lens or retinal lens 145 may be placed in direct contact with the cornea 151.

In a further embodiment the vibratory stimulus, the piezoelectric frequency generator 160, is placed in contact with the eyelid. In a further embodiment the piezoelectric frequency generator 160 is placed in contact with skin over one of the bones of the orbit or skull 154. The piezoelectric frequency generator 160 is capable of producing a specific frequency or a range of frequencies as required optimizing data integrity. In a further embodiment, the piezoelectric frequency generator 160 produces a range of frequencies from 0 to 2000 Hertz.

In a further embodiment, a force transducer 161 is attached to the piezoelectric generator 160 at the point of contact with the eye or the skin to monitor the input force spectrum in order to determine the frequency response function of the tissue of the eye 150 selected for measurement.

In a further embodiment, the piezoelectric frequency generator 160 is held in the hand of the subject whose eye 150 is to be measured. In a further embodiment the piezoelectric frequency generator 160 is placed in close proximity to the eye 150 but does not touch the eye 150 or any part of the subject. In a further embodiment, an audio speaker 162 attached to a frequency generator 163 is used to produce the vibratory stimulus.

In a further embodiment, a stream of modulated air pressure 164 is directed at the eye from an air source 165 to generate the vibratory stimulus.

In a further embodiment the curvature of the sclera or the velocity of the cornea and/or sclera at a single frequency is used to determine intraocular pressure.

In a further embodiment the average root mean square (RMS) power is determined by taking the square root of the integral of the velocity values of the tissue squared over a given frequency range to determine the intraocular pressure.

In a further embodiment the velocity of the tissue of the eye is used to calculate the stress on the tissue.

In a further embodiment the average RMS power is determined by taking the square root of the integral of the velocity values of the tissue squared over a given frequency range to calculate the stress on the tissue.

The curvature of the sclera, and therefore the intraocular pressure may be determined by a number of methods in accord with the present invention including but not limited to three measurement beams 122 used with a fourth reference beam 121, or three measurement beams 122 but no reference beam 121, but instead use the points of contact of the laser beams with the sclera for getting curvature which could incorporate a photographic detection method, or employ a scanning laser beam with a reference beam to obtain a profile of the scleral surface. Here a laser with a wavelength that is transmitted by the sclera will get a complete profile of the scleral surface and thickness, or utilize a laser of a wavelength that is not transmitted by the sclera which can be employed to provide just the surface of the sclera.

Preferably for measurement of the curvature of the sclera 152, data from three or more positional points 152a, 152b, 152c on the sclera 152 are employed. These multiple positional points may be obtained by a variety of methods including but not limited to use of a large beam 122, rapidly scanning a single beam 122, or further splitting beam 120 into a plurality of measuring beams 122, preferably at least three beams. By providing one measuring beam 122 that covers a generally greater surface area than the single beam utilized above, or by rapidly scanning a single measuring beam 122, or utilizing three measuring beams 122, a plurality of positional points 152a, 152b and 152c may be measured within the covered surface area.

In the preferred embodiment for detecting a plurality of positional points, the laser beam 120 is split into one reference beam 121 and three measuring beams 122. The reflection of these beams at position points 152a, 152b and 152c may be captured by a photographic device 130 or by using interference data to determine the three positional points on the surface of the sclera to be used to determine the curvature.

In a further embodiment for measuring the curvature of the sclera, lasers that have wavelengths that are not transparent to the sclera could be used for the surface points and wavelengths that are transparent to the sclera could be used for. obtaining the scleral profile from which the scleral radius of curvature is calculated.

The laser eye velocitometer apparatus 10 makes it possible to obtain precise, accurate and highly reproducible objective measurements of scleral curvature and the elastic properties of ocular tissues under normal conditions (in both developing and mature eyes) and in disease processes. These properties are utilized to determine the true intraocular pressure and the elastic characteristics of the ocular tissues, including: cornea, sclera, iris, pupillary sphincter, lens capsule, crystalline lens, optic nerve, lamina cribrosa, retinal blood vessels, preretinal membranes, intravitreal membranes, Bruch's membrane, choroidal vessels and retinal tissues and all of their interfaces.

Glaucoma is a one the most common causes of irreversible blindness in the world. Elevated ocular pressure within the eye is the major risk factor in the damage to the optic nerve that results in glaucoma and can lead to blindness. Ophthalmologists and optometrists measure intraocular pressure of their patients and visually assess the appearance of the optic nerve in order to detect ocular hypertension and any visible damage thereby to the optic nerve. The laser eye velocitometer 10 of the present invention can precisely and accurately measure intraocular pressure to objectively assess the vulnerability of the optic nerve to intraocular pressure. The laser eye velocitometer 10 uses scleral curvature and/or the velocity and resonant frequencies of the surface of the cornea, sclera, and optic nerve to characterize the intraocular pressure and elasticity characteristics of the optic nerve head surface in response to a vibratory stimulus. The scleral curvature and the velocity and resonant frequencies of the tissues within the optic nerve relate to their susceptibility to damage. Moreover, change is such that scleral curvature and/or velocities may reflect progression of glaucomatous damage. By measuring the scleral curvature and/or velocity of these tissues ocular hypertensive patients who are most vulnerable to glaucomatous damage can be identified. Currently there is no in vivo device that can measure scleral curvature or the optic nerve elastic characteristics.

### Age-Related Macular Degeneration

Hemorrhagic age-related macular degeneration (ARMD) is a major cause of blindness in the elderly. There are new medications that may be able to prevent and/or slow the progression of hemorrhagic ARMD, especially if the disease can be detected in its initial stages. One of the prodromal signs of ARMD is a change in the focal elasticity of a membrane located below the retina, Bruch's membrane. The laser eye velocitometer device 10 can quantify the early elastic changes in Bruch's membrane, before vascular abnormalities are induced, in order to identify those patients who are at risk for subsequent hemorrhagic ARMD. Identification of these sites within suspect eyes can offer both the opportunity for careful serial reevaluation and prompt preventive therapy.

### Diabetic Retinopathy

As a result of the significant increase in diabetes in the general population, diabetic retinopathy has become a major cause of blindness. Diabetic retinopathy begins as an alteration in the microvasculature of the retina. The laser eye velocitometer 10 can detect the early changes in the tissue of the retinal capillaries. Such diabetic individuals can be promptly treated with new medications that can slow or reverse these retinal vascular changes and prevent blindness.

### Presbyopia—Accommodation

Accommodation is the ability of the eye to focus at multiple distances and occurs as a result of a change in shape of the crystalline lens. This change in lenticular shape is a consequence of ciliary muscle contraction, which alters the stress applied to the lens. The laser eye velocitometer 10 can objectively measure lenticular stress and therefore accurately and precisely measure accommodative amplitude.

### EXAMPLE 1

An example illustrates that a correlation is achieved between the intraocular pressure and the velocity of eye tissue by using the apparatus and method of the present invention.

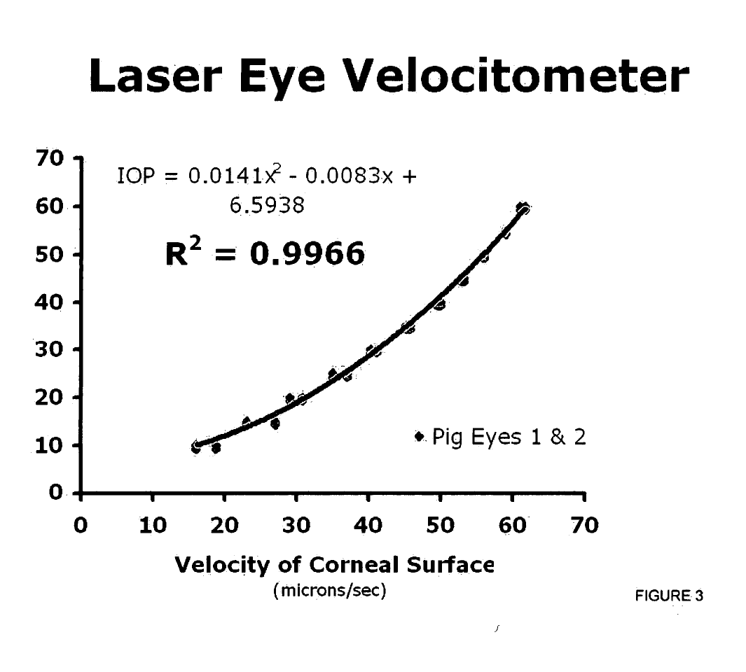

An apparatus of the type illustrated in FIG. 1 was used to project laser emissions on the sclera of pig eyes and detect the backscattered light for comparative analysis. In this study the intraocular pressure was varied while a vibratory stimulus was applied to the sclera. The study shows in FIG. 3 that the intraocular pressure had a highly significant correlation with the velocity of the surface of the cornea. Thus the apparatus and method of the present invention precisely, accurately and objectively measures intraocular pressure.

### EXAMPLE 2

An example illustrates that a correlation is achieved between the intraocular pressure and the scleral curvature of the eye by using the apparatus and method of the present invention.

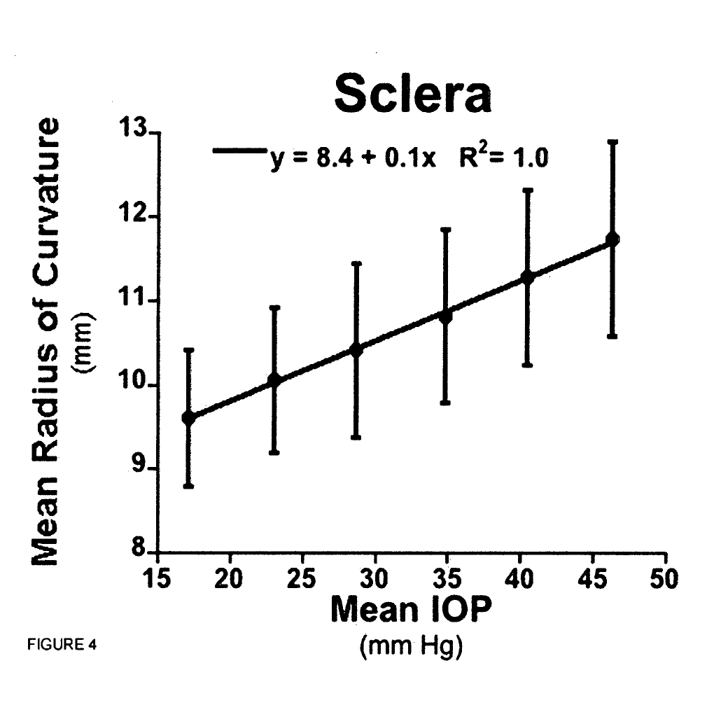

An apparatus of the type illustrated in FIG. 1 was used to project laser emissions onto the eye. A laser beam was divided into three beams and directed at the sclera of five pig eyes. An interferometric image of the sagittal sections of the sclera of the pig eyes was obtained on the sclera and detected the backscattered light for comparative analysis. Intraocular pressure was monitored manometrically. No vibratory stimulus was applied to the eyes. The change in position of the three reflected laser beams and the sagittal profile of the sclera were used to quantify scieral curvature. The study shows in FIG. 4 that there is a direct linear relationship between scleral curvature and intraocular pressure. Thus the apparatus and method of the present invention precisely, accurately and objectively measures intraocular pressure by scleral curvature.

Although the present invention has been described with an exemplary embodiment, various changes and modifications may be suggested to one skilled in the art. It is intended that the present invention encompass such changes and modifications as fall within the scope of the appended claims.

What is claimed is:

1. A method of determining the velocity and resonant frequency of tissues of the eye comprising:
a. Stimulating eye tissue to vibrate;
b. Dividing a beam of laser light into a reference beam and a measurement beam;
c. Directing said reference beam to a detector;
d. Directing said measurement beam to said stimulated eye tissue;
e. Capturing light of said measurement beam reflected from said tissue;
f. Directing said captured light to a detector;
g. Comparing in the detector output the phase and frequency shift of said reference beam and said measurement beam;
h. Calculating said velocity and said resonant frequency of said tissue;

2. The method of claim 1 wherein said resonant frequency of said stimulated tissue correlates to intraocular pressure;

3. The method of claim 1 wherein said resonant frequency of said stimulated tissue correlates to tissue stress;

4. The method of claim 1 wherein said stimulus generator is a piezoelectric frequency generator;

5. The method of claim 1 wherein said stimulus generator is a piezoelectric frequency generator directly contacting the cornea or sclera;

6. The method of claim 1 wherein said stimulus generator is a piezoelectric frequency generator directly contacting the eyelid;

7. The method of claim 1 wherein said stimulus generator is a piezoelectric frequency generator directly contacting the skin of one of the bones of the head;

8. The method of claim 1 wherein said stimulus generator is a piezoelectric frequency generator held in the hand or placed on any other part of the body of the subject;

9. The method of claim 1 wherein said stimulus generator is a stream of air;

10. The method of claim 1 wherein said stimulus generator is an audio speaker;

11. The method of claim 1 wherein said stimulus generator is an audio speaker attached to a frequency generator;

12. The method of claim 1 wherein said stimulus generator produces a range of frequencies from 0 to 2000 Hertz;

13. The method of claim 1 wherein said stimulus generator produces a range of frequencies from 0 to 10 megahertz;

14. The method of claim 1 wherein said laser comprises a diode laser, a tunable laser, or a laser that emits a specific wavelength selected from the spectrum that extends from the ultraviolet to the infrared;

15. The method of claim 1 wherein said laser emits a specific wavelength with a narrow frequency;

16. The method of claim 1 wherein said laser comprises a helium-neon laser with a wavelength of 633 nanometers;

17. The method of claim 1 wherein said laser emits a wavelength of ten (1) nanometers per second;

18. The method of claim 1 wherein said laser beam, reference beam or measurement beam is directed through fiber optics;

19. The method of claim 1 wherein said measuring beam is directed through a biomicroscope (slit lamp), a surgical microscope or a stereo microscope;

20. The method of claim 1 wherein said measuring beam is directed to a retinal camera;

21. The method of claim 1 wherein the position of said measuring beam is controlled by steerable optics;

22. The method of claim 1 wherein the position of said reference beam is controlled by steerable optics;

23. The method of claim 1 wherein in the detector output the Doppler shift of the laser beam is correlated to the velocity of tissues of the eye;

24. The method of claim 1 wherein said measuring beam is directed at one tissue of the eye and the reference beam is directed at a different tissue of the eye to determine the differential velocity and differential resonant frequencies of the two different tissues of the eye;

25. The method of claim 1 to enable measurement of tissues within the eye, further comprising placing a concave or a +90 diopter lens between a biomicroscope and said eye surface;

26. The method of claim 1 to enable measurement of tissues within the eye, further comprising placing a gonio lens or a retinal lens in direct contact with said eye tissue;

27. The method of claim 1 wherein the velocity of the cornea at a single frequency is used to correlate to intraocular pressure;

28. The method of claim 1 wherein said resonant frequency of said stimulated tissue correlates to intraocular pressure diagnostic for disease, said disease comprising glaucoma;

29. The method of claim 1 wherein said resonant frequency of said stimulated tissue correlates to changes in said tissue diagnostic of disease, said disease comprising age related macular degeneration and diabetic retinopathy;

30. The method of claim 1 wherein the intraocular pressure is determined by correlation with the average root mean square further comprising taking the square root of the integral of the velocity values of the tissues squared over a given frequency range;

31. The method of claim 1 wherein the stress on said tissue is determined by correlation with the average root mean square further comprising taking the square root of the integral of the velocity values of the tissues squared over a given frequency range;

32. An apparatus for measuring the velocity and resonant frequencies of the tissues of the eye comprising,
a. A stimulus generator to stimulate said tissue;
b. A laser generating a beam of light, said beam of light divided into a reference beam and a measurement beam;
c. Said measurement beam directed to said stimulated tissue and light reflected from said stimulated tissue directed to a detector;
d. said reference beam directed to a detector; and
e. an apparatus to calculate said velocity and said resonant frequency of said stimulated tissue from the output of said reference beam detector and said measurement beam detector;

33. The apparatus of claim 32 wherein a plurality of measurement beams are utilized;

34. The apparatus of claim 32 wherein said laser emits a specific wavelength with a narrow frequency;

35. The apparatus of claim 32 wherein said stimulus generator is a piezoelectric frequency generator;

36. The apparatus of claim 32 wherein said stimulus generator is a stream of air;

37. The apparatus of claim 32 wherein said stimulus generator is an audio speaker attached to a frequency generator;

38. The apparatus of claim 32 wherein said apparatus directly contacts said tissue;

39. The apparatus of claim 32 wherein said stimulus generator produces a range of frequencies from 0 to 2000 Hertz;

40. The apparatus of claim 32 wherein said stimulus generator produces a range of frequencies from 0 to 10 megahertz;

41. The apparatus of claim 32 wherein said measuring beam is directed through a biomicroscope (slit lamp), or a surgical microscope or a stereo microscope;

42. The method of claim 32 wherein the position of said measuring beam is controlled by steerable optics;

43. The method of claim 32 wherein the position of said reference beam is controlled by steerable optics;

44. The apparatus of claim 32 wherein said measuring beam is directed to a retinal camera;

45. The apparatus of claim 32 wherein said measuring beam is directed at one tissue of the eye and the reference beam is directed at a different tissue of the eye to determine the differential velocity and differential resonant frequencies of the two different tissues of the eye;

46. The apparatus of claim 32 wherein said apparatus measures the intraocular pressure of the eye;

47. A method of measuring the material properties of tissue comprising:
a. Stimulating said tissue to vibrate;
b. Dividing a beam of laser light into a reference beam and a measurement beam;
c. Directing said reference beam to a detector;
d. Directing said measurement beam to said stimulated tissue;
e. Capturing light of said measurement beam reflected from said tissue;
f. Directing said captured light to a detector;
g. Comparing in the detector output the phase and frequency shift of said reference beam and said measurement beam;
h. Calculating the amplitude, frequency and velocity of the response of said tissue to said vibratory stimulus;

48. A method of determining the intraocular pressure of the eye comprising:
a. Stimulating scleral tissue of the eye to vibrate;
b. Dividing a beam of laser light into a reference beam and a plurality of measurement beams;
c. Directing said reference beam to a detector;
d. Directing said measurement beams to a plurality of position points on said stimulated scleral tissue;
e. Capturing light of said measurement beams reflected from said position points on said scieral tissue;
f. Directing said captured light from said position point to a detector;
g. Comparing in the detector output the phase and frequency shift of said reference beam and said measurement beams to calculate scleral curvature;
h. Correlating said scleral curvature to intraocular pressure;

49. A method of disease diagnosis of the eye comprising:
a. Stimulating eye tissue to vibrate;
b. Directing a laser beam to divide into a reference beam and a measurement beam;
c. Directing said reference beam to a detector;
d. Directing said measurement beam to said stimulated tissue;
e. Capturing light of said measurement beam reflected from said stimulated tissue;
f. Directing said captured light to a detector;
g. Comparing the phase and frequency shift of said reference beam and said measurement beam;
h. Calculating velocity and resonant frequency of said tissue;
i. Comparing said velocity and said resident frequency to values for healthy tissue and unhealthy tissue to diagnose disease;

50. The method of claim 49 wherein said disease comprises glaucoma, age related macular degeneration or diabetic retinopathy;
